(19) United States
(12) Patent Application Publication
(10) Pub. No.: US 2002/0100082 A1
(43) Pub. Date: Jul. 25, 2002

(54) Method for determining content of heterologous individual

(76) Inventors: Hiroshige Yamashita, Kusatsu-shi (JP); Ikunoshin Kato, Uji-shi (JP)

Correspondence Address: BROWDY AND NEIMARK, P.L.L.C., 624 NINTH STREET, NW, SUITE 300, WASHINGTON, DC, 20001-5303, US

(21) Appl. No.: 09/774,107

(22) Filed: Jan. 31, 2001

(30) Foreign Application Priority Data
Mar. 3, 2000 (JP) 58726/2000

(51) Int. Cl. A01H 1/00; C12N 15/82; C12N 15/87

(52) U.S. Cl. 800/294

(57) ABSTRACT

A method for quantitatively and precisely determining a content of a heterologous individual in a population of an organism is disclosed.

### BACKGROUND OF THE INVENTION

1. Field of the Invention

The present invention relates to a method for detecting a gene. Specifically, the present invention relates to a method for quantitatively and precisely determining a content of a heterologous individual in a population of an organism.

2. Description of Related Art

Genetically modified organisms (GMOs), which have genes modified to improve and/or alter the productivity or the quality of crops, have been developed. For example, crops in which genes are modified to confer resistance to noxious insects or herbicides, are commercially available (Wolfram Hemmer, Foods Derived from Genetically Modified Organisms and Detection Methods, February 1997, Agency BATS).

Due to the anxiety about the safety of the GMOs (see, for example, Losey, J. E. et al., Nature, 399:214 (1999)), separation of the GMOs from non-genetically modified organisms (non-GMOs) and/or indication of the use of GMO have been desired. In response, an identity preserved (IP) transport system is conducted in order to prevent the GMOs from contaminating into the non-GMOs. Furthermore, the Ministry of Agriculture, Forestry and Fisheries of Japan published a proposed standard for quality indication of GMOs under Japanese Agricultural Standard (JAS) Law on Nov. 29, 1999.

However, for example, the GMO and the non-GMO may be mixed together when the IP transport is not used. Alternatively, they may be mixed intentionally or accidentally even if the IP transport is used. Thus, development of quantitative examination techniques for guaranteeing the separation of the GMOs from the non-GMOs and determining their contents has been desired.

Methods which may be used for determining the content of a crop differing in a specific genotype (e.g., a GMO) in a crop test sample may be exemplified by a polymerase chain reaction (PCR) for specifically amplifying a nucleotide sequence and an enzyme-linked immunosorbent assay (ELISA) for immunologically detecting a protein encoded by a gene. However, as described below, it is difficult to precisely determine the content of the GMO in the crop test sample using these methods.

The ELISA is a method in which the binding between a protein of interest and an enzyme-labeled antibody directed to the protein of interest is quantitatively determined. It is required to prepare a sample containing proteins from the test sample to be tested in order to apply the ELISA to the examination for the crop test sample. However, it is difficult to prepare all proteins from a test sample with a defined efficiency. Therefore, it is considered that results from a determination method utilizing an ELISA may include much error and may be lower than the true values. Furthermore, if a processed food is to be tested, since proteins in the test sample may be denatured by heating or the like, the determination would become more difficult.

A PCR which is a method for amplifying a nucleic acid is used for a variety of purposes. In particular, since the PCR can specifically amplify a nucleic acid in large quantities, it is useful as a sensitive method for detecting a nucleic acid. For example, the PCR is used for detecting modified CryIA gene in maize (JP-A 11-266875) Although the PCR is generally used for qualitative detection, attempts have been made to quantify a nucleic acid of interest in a sample using a PCR. For example, a competitive PCR has been described. In the method, a PCR is conducted with the addition of a defined amount of a competitive template that is amplified in a similar manner with that of the nucleotide sequence of interest and results in a band with a size different from that of that for the nucleotide sequence of interest (e.g., Wang, A.M. et al., Proc. Natl. Acad. Sci. USA, Vol. 86, 9717-9721 (1989)). However, the operation of this method is complicated. Furthermore, it is difficult to obtain precise measurements using this method. As described above, no quantitative and precise determination method utilizing a PCR which can be applied to the determination of a content of a GMO in a test sample is known.

### SUMMARY OF THE INVENTION

The present invention provides a method for quantitatively and precisely determining a content of a heterologous individual in a population of an organism.

The present inventors have intensively studied in order to accomplish the above-mentioned objects. As a result, the present inventors have found that a content of a heterologous individual in a population of an organism can be quantitatively and precisely determined by conducting quantitative PCRs for both of a target gene and a control gene using DNAs extracted from plural samples obtained from a population of an organism or a processed product from the population of the organism as templates; calculating the content of the heterologous individual in the population of the organism based on the amounts of the amplification products for the respective genes; and determining the confidence of the calculated content by statistically analyzing the determined results obtained independently for plural samples. Thus, the present invention has been completed.

The present invention provides a method for quantitatively determining a content of a heterologous individual in a population of an organism, characterized in that the method comprises:

a) obtaining two or more samples from a population of an organism or a processed product from the population of the organism;

b) extracting DNAs from the samples;

c) preparing reaction mixtures each containing one of the extracted DNAs, primers specific for a target gene and primers specific for a control gene;

d) subjecting the reaction mixtures to quantitative PCRs to determine amounts of amplification products specific for the target gene and amounts of amplification products specific for the control gene;

e) determining a content of a heterologous individual in the population of the organism based on the amounts of the amplification products specific for the target gene and the amounts of the amplification products specific for the control gene; and

f) determining the confidence of the determined content by statistical analysis.

### DETAILED DESCRIPTION OF THE INVENTION

As used herein, the term heterologous individual refers to an organism which is present in a population of an organism and differs in a specific genotype. The difference in the specific genotype may be due to a gene that specifically exists in a specific variety, an artificially introduced gene or an artificially modified endogenous gene.

The artificially introduced genes include a gene encoding a foreign protein, and a gene encoding a promoter, an enhancer, an antisense nucleic acid or the like for controlling the expression of an endogenous gene.

Examples of the heterologous individuals include a specific variety in a test sample in which plural varieties are mixed together, an individual infected with a pathogen such as a virus and a genetically modified organism (GMO) into which a non-natural gene is introduced or in which an endogenous gene is modified.

The present invention is mainly described with respect to the determination of a content of a GMO in a crop test sample hereinafter. However, the present invention can be applied to all organisms including eukaryotes and prokaryotes. Using the method of the present invention, a content of a specific variety in a test sample in which plural varieties are mixed together can be determined. For example, some of expensive rice products commercially available under the brand name “Koshihikari” contain low-priced rice varieties other than the variety “Koshihikari”. Determination of the content of the variety “Koshihikari” in such a rice product is important for the indication of a crop.

The populations of the organisms used for the present invention include the population of the organism itself and a product processed therefrom. If the population of the organism is a crop, an unprocessed crop and a processed food made from the crop as a raw material can be used for the present invention. The unprocessed crops are any crops which can be genetically engineered and include, but are not limited to, soybean, maize, rice, wheat, barley, tomato, pumpkin, potato, sweet potato, cotton, rapeseed and beet. The processed foods are any foods which are processed from the above-mentioned unprocessed crops and include, but are not limited to, tofu (bean curd), tofu-related food (fried bean curd, dried bean curd, soybean milk, sheet of dried bean curd, etc.), defatted soybean, soybean flour, unpurified soybean proteins, corn grits, corn flour, cornstarch, popcorn and snack food. The processed foods also include dried crops. A content of a heterologous individual in a processed food refers to a content of the heterologous individual contained in a raw material for the processed food.

An amount of a DNA contained in a processed food that has been subjected to a step of heating, enzymatic digestion, purification, fermentation or the like may be reduced, and/or such a DNA may be degraded. The ability of the method of the present invention to determine the content of a GMO in such a processed food is judged on the basis of the results for amplification of a control gene as described below.

As used herein, a genetically modified organism (GMO) refers to a crop modified by introducing a foreign gene or modifying an endogenous gene. Examples of the GMOs include, but are not limited to, the following: a crop into which modified 5-enolpyruvoylshikimate-3-phosphate synthase (EPSPS) protein gene is introduced to confer resistance to a herbicide glyphosate (product name “Roundup”); a crop into which phosphinothricin acetyltransferase (pat) gene from Streptomyces viridochrimogenes is introduced to confer resistance to a herbicide glufosinate (product name “Basta”); a crop into which bar gene from Streptomyces hygroscopicus, a homologue of pat gene, is introduced to confer resistance to a herbicide glufosinate (product name “Basta”); and a crop into which CryIA gene or CryIIIA gene from Bacillus thuringiensis is introduced to confer resistance to noxious insects.

Furthermore, GMOs with modified endogenous genes include one in which a mutation such as substitution, deletion, insertion or addition is introduced into a specific endogenous structural gene to alter the activity of the product of the gene and one in which a mutation such as substitution, deletion, insertion or addition is introduced into a specific endogenous regulatory gene (promoter, enhancer, etc.) to alter the expression of the gene under control of the regulatory gene.

The samples of the present invention are obtained from a population of an organism suspected to contain a heterologous individual, a processed product made from the population of the organism as a raw material and a preparation obtained by processing the population of the organism or the processed product. For example, if the population of the organism is a crop such as maize or soybean, a sample is obtained from a preparation obtained by grinding the crop to homogeneity (for example, about 2 kg of the crop corresponding to about 10,000 grains is ground in order to accomplish a detection limit of 0.01%). If the subject to be determined is a homogeneous processed food, such processing is not required.

Two or more samples are obtained from one test sample in the method of the present invention. In general, more precise measurements are obtained by using more number of samples. On the other hand, it is preferable to reduce the number of samples in order to make the operation convenient. For example, 2 to 30, preferably 3 to 20, more preferably 5 to 10, most preferably 4 to 8 samples are obtained from one test sample. The amount of the sample is determined such that it is suitable for the extraction of a DNA. For example, if soybean or corn powder, or dried tofu powder is used, 0.5 to 1 g of a sample is collected.

A DNA extraction method suitable for the sample to be used is selected. For example, a known DNA extraction method can be used to extract genomic DNAs from corn powder and soybean powder. For example, if DNAs from a large number of samples are to be extracted, a DNA extraction robot BIOROBOT9604 (Qiagen) and a DNA extraction kit for this robot, or an automated instrument GENEEXTRACTOR TA100 (Takara Shuzo) and an extraction reagent kit GENEEXTRACTIN BC kit (Takara Shuzo) can be used. Also, a DNA synthesized according to a known method from an RNA extracted according to a known method can be used for the present invention.

As used herein, a target gene refers to a gene to be detected that is not present in an organism other than a heterologous individual (e.g., a non-genetically modified organism (a non-GMO)) but is present in the heterologous individual (e.g., a GMO), or of which the sequence in a heterologous individual is different from that in an organism other than the heterologous individual. Examples of the target genes include the above-mentioned foreign genes introduced in genetically modified organisms and modified endogenous genes. Preferably, the nucleotide sequence of the target gene is known. If the nucleotide sequence of the target gene is unknown, the nucleotide sequence of the gene can be determined by using recombinant DNA techniques well known in the art including, for example, PCR, cloning and sequencing. Examples of the target genes include, but are not limited to, pat gene, modified EPSPS protein gene, CryIA gene, bar gene and CryIIIA gene.

As used herein, a control gene refers to any one of genes that are present in both of a heterologous individual (e.g., a GMO) and an organism other than the heterologous individual (e.g., a non-GMO). Preferably, the nucleotide sequence of the control gene is known. Examples of the control genes include, but are not limited to, zein gene, actin protein gene and lectin gene.

The quantitative PCR in the method of the present invention is a PCR that enables the quantitative determination of a content of a heterologous individual in a population of an organism based on amounts of amplification products from a target gene and a control gene. For example, the quantitative determination can be accomplished by conducting a PCR under reaction conditions that enable the quantitative determination, subjecting the amplification products to electrophoresis and determining the amounts based on the intensities of the bands corresponding to the amplification products. It is preferable to use an instrument for PCR that can determine the amounts of amplification products over time (a real- time PCR instrument) in order to carry out the quantitative determination more conveniently and precisely. PCR instruments such as LightCycler (Roche) and ABI Sequence Detector PRISM 7700 (Perkin-Elmer Biosystems) are commercially available. Methods for conducting the real- time PCR include TaqMan method (Japanese Patent no. 2825976), a hybridization method and a method in which an intercalator such as SYBR Green I Nucleic Acid Gel Stain (BMA) is used. All of these methods can be used for the present invention.

A reaction mixture to be subjected to a quantitative PCR contains a DNA extracted from a sample, primers specific for a target gene and primers specific for a control gene. Methods for designing and preparing primers are known in the art. The reaction mixture may further contain a heat-resistant DNA polymerase, dNTPs, a buffer and other additives. Such components are suitably selected depending on the instrument for PCR and the kit to be used or the like. The reaction mixture may contain a labeled probe for detecting an amplification product.

The content of the heterologous individual in the population of the organism is determined based on the amount of the amplification product specific for the target gene and the amount of the amplification product specific for the control gene in the method of the present invention. In one embodiment, initial amounts of DNAs as templates for both of the target gene and the control gene contained in one sample are calculated based on the amounts of the respective amplification products using calibration curves. Determination for samples is conducted under the same conditions (including the combination of the amplification primers and the detection probe) as those used for making the calibration curves.

First, a DNA is extracted from a sample derived from a positive population of an organism that exclusively contains a heterologous individual such as a GMO. The DNA is serially diluted to prepare dilutions. A PCR is carried out using one of the dilutions as a template for each of a target gene and a control gene. Calibration curves for initial amounts of DNAs as templates versus amounts of amplification products are made based on the results. Similarly, samples to be determined are then subjected to DNA extraction and PCR to determine the amounts of amplification products. The initial amounts of DNAs as templates are determined for the respective genes using the calibration curves made as described above. The ratio (percentage) of the initial amount of DNA as a template for the target gene to the initial amount of DNA as a template for the control gene is calculated to determine the content of the heterologous individual (the GMO) in the population of the organism. The initial amount of DNA as template thus determined for the sample is a relative value, which may vary depending on factors such as the DNA extraction efficiencies which may differ among the DNAs as templates for making calibration curves and the DNAs from the samples, the presence or the amount of an inhibitor of PCR which may be contained in the extracted DNA, and the like. However, such factors contribute to the results for both of the control gene and the target gene in the same manner. Therefore, the variation does not influence the finally determined content if the content of the heterologous individual in the population of the organism is determined by calculating the ratio of the amounts of the two DNAs as templates.

When fine powder obtained by grinding a crop test sample is used as a sample, errors due to sampling may be included in measurements even if the powder is thoroughly mixed to homogeneity. Errors may be similarly included when an apparently homogeneous processed food is used as a subject to be determined. Furthermore, errors may also be included in an extraction step and a PCR step. In the method of the present invention, the confidence of the measurements that include such errors is guaranteed by conducting independent determination for each of plural samples and statistically analyzing the results. Statistical analyses which can be applied to the present invention are known in the art. For example, an interval defined by a least upper bound and a greatest lower bound that guarantees a given (e.g., 99%) confidence of the measurements can be determined using interval estimation. Before the present invention, no such statistical analysis is applied to a method for quantitatively determining a content of a heterologous individual in a population of an organism.

### EXAMPLES

The following Examples further illustrate the present invention in detail but are not to be construed to limit the scope thereof.

### Example 1

### Determination of Content of Genetically Modified Maize

A GMO, maize of T14/25 line (product name: VARIETY8539, Garst Seed Company, hereinafter referred to as T14/25), was used as positive maize. T14/25 contains phosphinothricin acetyltransferase (pat) gene from Streptomyces viridochrimogenes (product name: Liberty Link, Hoechst/AgrEvo), of which the copy number on the genome is known to be 1. Its sequence and copy number are described in documents submitted to the Ministry of Health and Welfare of Japan by AgrEvo The information contained in the documents is available from Japanese Food Hygiene Association. Zein protein is ubiquitously present in maize, and the nucleotide sequence of the gene encoding this protein is known (Kirihara, J. A. et al., Mol. Gen. Genet, 211:477-484 (1988)). The content of T14/25 in a maize test sample was determined by determining the ratio of amounts of pat gene as a target gene and zein gene as a control gene.

Maize without a GMO (hereinafter referred to as GMO Free) was used as negative maize. Maize test samples that contain 2 or 6% by weight of T14/25 in the GMO Free maize (designated as A and B, respectively) were prepared. The maize test samples A and B were prepared as shown in Table 1.

TABLE 1

| Maize test sample | A | B |
|---|---|---|
| T14/25 | 40 g | 120 g |
| GMO Free | 1960 g | 1880 g |
| Total weight | 2000 g | 2000 g |
| Content of T14/25 | 2% | 6% |

Each of the mixed maize test sample was ground to fine powder using a cutter mixer 5.5 (DITO SAMA, France). 8 samples each containing 1 g of the powder were obtained from the ground maize test sample A or B. Genomic DNAs were independently extracted therefrom. A DNA extraction robot BIOROBOT9604 (Qiagen) and a DNA extraction kit for the robot were used for extracting the genomic DNAs. The extracted DNA was dissolved in the AE Buffer attached to the kit to obtain 100 μl of a DNA solution.

Powder obtained by grinding a test sample that exclusively contains T14/25 was used as a positive sample. A genomic DNA was extracted from the sample as described above. The thus obtained DNA was serially diluted with sterile distilled water to prepare five dilutions (the original solution, a 10-fold dilution, a 100-fold dilution, a 1,000-fold dilution and a 10,000-fold dilution).

A PCR for amplifying pat gene or zein gene was carried out using one of the dilutions as a template. Calibration curves for the respective genes were made based on the results. A pair of primers for amplifying pat gene, pat FP and pat RP (SEQ ID NOS: 1 and 2), and a fluorescence-labeled probe (SEQ ID NO: 3) were used for the amplification and detection of pat gene. A pair of primers for amplifying zein gene, Zein FP and Zein RP (SEQ ID NOS: 4 and 5), and a fluorescence-labeled probe (SEQ ID NO: 6) were used for the amplification and detection of zein gene. FAM and TAMRA (both from Glen Research) were added at the 5′-termini and the 3′-termini of the two fluorescence- labeled probes, respectively. Table 2 shows the composition of the reaction mixture for amplification reaction.

TABLE 2

| | | |
|---|---|---|
| DNA as template | 5 | μl |
| Primer 1 (10 pmol/μl) | 1.5 | μl |
| Primer 2 (10 pmol/μl) | 1.5 | μl |
| Fluorescence-labeled probe (2 pmol/μl) | 5 | μl |
| TaqMan 2 × PCR Master Premix | 25 | μl |
| Sterile distilled water | 12 | μl |
| Reaction volume | 50 | μl |

A PCR was carried out for each of the dilutions in duplicate. The reaction was carried out in a 96-well plate using ABI Sequence Detector PRISM 7700 (Perkin-Elmer Biosystems) according to TaqMan PCR method. Amplification of the target gene (pat gene) and the control gene (zein gene) was recorded over time to make calibration curves for these genes.

Next, a PCR was carried out using one of the genomic DNAs extracted from the eight samples from the maize test sample A as well as those from the maize test sample B as a template in a similar manner. The relative initial amounts of DNAs as templates in the samples were calculated for the target gene (pat gene) and the control gene (zein gene) ((a) and (b) in Tables 3 and 4, respectively) using the calibration curves. Percentage of the target gene (pat gene) to the control gene (zein gene) ((a)/(b)×100) was calculated for each of the samples. Furthermore, a greatest lower bound and a least upper bound of 99% confidence were determined according to interval estimation method based on the relative percentages obtained for the eight samples for each of the maize test samples A and B. The results are shown in Tables 3 and 4.

TABLE 3
Maize test sample A (containing 2% of T14/25)

| | Amount of | Amount of | |
|---|---|---|---|
| | target gene | control gene | (a)/(b) × 100 |
| Sample no. | (a) | (b) | (%) |
| 1 | 4.78 | 206 | 2.33 |
| 2 | 5.04 | 147 | 3.44 |
| 3 | 7.10 | 321 | 2.21 |
| 4 | 6.92 | 210 | 3.30 |
| 5 | 5.22 | 324 | 1.61 |
| 6 | 6.27 | 278 | 2.26 |
| 7 | 5.97 | 260 | 2.30 |
| 8 | 6.42 | 278 | 2.31 |

| | | |
|---|---|---|
| | Mean (%) | 2.47 |
| | Standard deviation (%) | 0.6042 |
| | Coefficient of variation (%) | 24.5 |
| Interval of | Greatest lower bound (%) | 1.5 |
| 99% confidence | Least upper bound (%) | 3.5 |

TABLE 4
Maize test sample B (containing 6% of T14/25)

| | Amount of | Amount of | |
|---|---|---|---|
| | target gene | control gene | (a)/(b) × 100 |
| Sample no. | (a) | (b) | (%) |
| 1 | 19.1 | 281 | 6.79 |
| 2 | 21.9 | 320 | 6.83 |
| 3 | 14.7 | 252 | 5.82 |
| 4 | 15.4 | 256 | 6.02 |
| 5 | 22.0 | 333 | 6.61 |
| 6 | 18.5 | 259 | 7.13 |
| 7 | 19.9 | 269 | 7.38 |
| 8 | 19.4 | 278 | 6.97 |

| | | |
|---|---|---|
| | Mean (%) | 6.69 |
| | Standard deviation (%) | 0.5332 |
| | Coefficient of variation (%) | 8.0 |
| Interval of | Greatest lower bound (%) | 5.8 |
| 99% confidence | Least upper bound (%) | 7.6 |

For each of the maize test samples A and B, the content 2% or 6%) of T14/25 added was between the greatest lower bound and the least upper bound determined based on the measurements according to interval estimation.

### Example 2

### Determination of Content of Genetically Modified Soybean

Soya Bean Powder SB-Set (Fluka, individual sample number 1673, lot and filling code 92683/1) comprising test samples each containing a GMO, Roundup Ready (Monsanto), at a concentration of 0, 0.1, 0.5 or 2% in soybean without a GMO was used as positive maize. This is standard soybean powder approved by Institute for Reference Materials and Measurements (IRMM).

Roundup Ready contained in the sample contains modified (CP4) EPSPS protein gene from Agrobacterium tumefaciens CP4 strain, of which the copy number on the genome is known to be 1. Actin protein is ubiquitously present in soybean, and the nucleotide sequence of the gene encoding this protein is known (Shah, D. M. et al., Proc. Natl. Acad. Sci. USA, Vol. 79, 1022-1026 (1982)). The contents of Roundup Ready in the test samples from Soya Bean Powder SB-Set were determined by determining the ratio of amounts of modified (CP4) EPSPS protein gene as a target gene and actin gene as a control gene.

Four samples were obtained from 0.5 g of one of the four test samples from Soya Bean Powder SB-Set. Genomic DNAs were independently extracted therefrom. A DNA extraction robot BIOROBOT9604 (Qiagen) and a DNA extraction kit for the robot were used for extracting the genomic DNAs. The extracted DNA was dissolved in the AE Buffer attached to the kit to obtain 100 μl of a DNA solution.

Agarose gel electrophoresis of the extracted DNAs for the confirmation of their states revealed a ladder-like pattern, indicating that the DNA of the soybean was enzymatically digested due to apoptosis during storage or in the course of preparation of soybean powder. When PCR is carried out using such a DNA as a template, amplification efficiency may vary among samples even if the same primers are used, thus making precise quantification difficult.

Powder obtained by grinding a test sample that exclusively contains Roundup Ready was used as a positive sample. A genomic DNA was extracted from the sample as described above. The thus obtained DNA was serially diluted with sterile distilled water to prepare five dilutions (the original solution, a 10-fold dilution, a 100-fold dilution, a 1,000-fold dilution and a 10,000-fold dilution).

A PCR for amplifying modified EPSPS protein gene or actin gene was carried out using one of the dilutions as a template. Calibration curves for the respective genes were made based on the results. A pair of primers for amplifying modified EPSPS protein gene, CP4 FP and CP4 RP (SEQ ID NOS: 7 and 8), and a fluorescence-labeled probe (SEQ ID NO: 9) were used for the amplification of modified EPSPS protein gene. A pair of primers for amplifying actin gene, actin FP and actin RP (SEQ ID NOS: 10 and 11), and a fluorescence-labeled probe (SEQ ID NO: 12) were used for the amplification of actin gene. FAM and TAMRA were added at the 5′-termini and the 3′-termini of the two fluorescence-labeled probes, respectively. Table 5 shows the composition of the reaction mixture for amplification reaction.

TABLE 5

| | | |
|---|---|---|
| DNA as template | 5 | μl |
| Primer 1 (10 pmol/μl) | 1.5 | μl |
| Primer 2 (10 pmol/μl) | 1.5 | μl |
| Fluorescence-labeled probe (2 pmol/μl) | 5 | μl |
| TaqMan 2 × PCR Master Premix | 25 | μl |
| Sterile distilled water | 12 | μl |
| Reaction volume | 50 | μl |

A PCR was carried out for each of the dilutions in duplicate. The reaction was carried out in a 96-well plate using ABI Sequence Detector PRISM 7700 (Perkin-Elmer Biosystems) according to TaqMan PCR method. Amplification of the target gene (modified EPSPS protein gene) and the control gene (actin gene) was recorded over time to make calibration curves for these genes.

Next, a PCR was carried out using one of the genomic DNAs extracted from the four samples from each of the four test samples from Soya Bean Powder SB-Set as a template in a similar manner. The relative initial amounts of DNAs as templates in the samples were calculated for the target gene (modified EPSPS protein gene) and the control gene (actin gene) ((a) and (b) in Table 6, respectively) using the calibration curves. Percentage of the target gene (modified EPSPS protein gene) to the control gene (actin gene) ((a)/(b)×100) was calculated for each of the samples. Furthermore, a greatest lower bound and a least upper bound of 99% confidence were determined according to interval estimation method for each of the four test samples from Soya Bean Powder SB-Set. The results are shown in Tables 6.

TABLE 6

| IRMM sample | | | | | | | | | |
|---|---|---|---|---|---|---|---|---|---|
| Indi- | | | | | | | | | |
| cated | Sample | | | (a)/(b) | | | | IC (99%)⁵ | |
| conc. | no. | (a)¹ | (b)² | (%) | Mean | SD³ | CV⁴ | GLB⁶ | LLB⁷ |
| 0% | 1 | 0.007 | 217 | 0.00% | | | | | |
| | 2 | N.D. | 109 | N.D. | | | | | |
| | 3 | N.D. | 114 | N.D. | | | | | |
| | 4 | N.D. | 88.9 | N.D. | — | — | — | — | — |
| 0.10% | 1 | 0.441 | 299 | 0.15% | | | | | |
| | 2 | 0.191 | 337 | 0.06% | | | | | |
| | 3 | 0.261 | 295 | 0.09% | | | | | |
| | 4 | 0.279 | 251 | 0.11% | 0.10% | 0.0004 | 0.3788 | 0 | 0.2 |
| 0.50% | 1 | 0.754 | 204 | 0.37% | | | | | |
| | 2 | 0.826 | 349 | 0.24% | | | | | |
| | 3 | 1.41 | 241 | 0.59% | | | | | |
| | 4 | 0.811 | 440 | 0.18% | 0.34% | 0.0018 | 0.5196 | 0 | 0.8 |
| 2% | 1 | 4.41 | 281 | 1.57% | | | | | |
| | 2 | 4.19 | 191 | 2.19% | | | | | |
| | 3 | 4.13 | 330 | 1.25% | | | | | |
| | 4 | 3.11 | 629 | 0.49% | 1.38% | 0.0071 | 0.5132 | 0 | 3 |

N.D.: below detection limit;
(a)¹: amount of target gene;
(b)²: amount of control gene;
SD³: standard deviation;
CV⁴: coefficient of variation;
IC (99%)⁵: interval of 99% confidence;
GLB⁶: greatest lower bound;
LLB⁷: least upper bound.

For each of the four test samples from Soya Bean Powder SB-Set, the content (0, 0.1, 0.5 or 2%) of Roundup Ready was between the greatest lower bound and the least upper bound determined based on the measurements according to interval estimation.

### Example 3

### Content of Genetically Modified Soybean in Tofu Test Sample

It was examined whether or not the content of GMO soybean can be determined for tofu of which the main raw material is soybean. A raw material soybean standard containing 4% of GMO was prepared using Roundup Ready (Monsanto) as positive soybean as described in Example 2. The raw material soybean standard was ground as described in Example 2 and soaked in sterile water overnight. The supernatant was heated (at about 80° C.), and a commercially available coagulating agent (bittern) was added thereto. The mixture was gently mixed until it solidifies as tofu. Solidified tofu was filtered through gauze to remove water. Thus, a tofu test sample with a known content of GMO was obtained.

Dried tofu powder was obtained by lyophilizing the tofu test sample to completely remove water. Genomic DNAs were extracted from four samples each containing about 0.5 g of powder.

0.5 g of the powder sample was placed in a centrifugation tube. 3.5 ml of an extraction buffer [10 mM tris-hydrochloride (pH 8.0), 150 mM NaCl, 2 mM EDTA, 1% SDS] containing proteinase K at a concentration of 20 mg/ml and 400 μl of 5M guanidine were added thereto. The suspension was heated at 58° C. for 1 hour. 300 μl of a supernatant obtained by centrifugation at 3000 rpm for 5 minutes was recovered. 100 μl of a solution [10 mM EDTA, 50 mM tris-hydrochloride (pH 7.5)] containing RNase A at a concentration of 100 μg/pl was added thereto. 200 μl of a 0.2% SDS solution was then added to the suspension. The suspension was mixed by shaking for 5 minutes. 1 ml of a mixture prepared by mixing 63 volumes of a nucleic acid absorbent suspension [50 mg/ml silica gel particle (particle diameter: 5 μm, Fuji Sylisia), 7 M guanidine hydrochloride, 2 mM EDTA, 10 mM tris-hydrochloride (pH 7.5)] and 37 volumes of ethanol was added thereto. The mixture was further shaken for 5 minutes.

The mixture was centrifuged, the supernatant was removed, and the precipitate was suspended in 300 μl of a washing solution [50%(v/v) ethanol, 100 mM NaCl, 2.5 mM EDTA, 10 mM tris-hydrochloride (pH 7.5)]. The suspension was transferred to SUPREC-01 (Takara Shuzo), and centrifuged at 12000 rpm for 5 minutes for washing. After repeating the washing procedure once more in a similar manner except that 200 μl of the washing solution was used, the precipitate was suspended in 100 μl of TE buffer which had been warmed to 70° C. After centrifugation, supernatant was recovered. The resulting supernatant was used as a DNA solution in the following steps.

Agarose gel electrophoresis of the extracted DNA for the confirmation of its state revealed that high molecular weight DNA migrated as a smear, indicating that the DNA contained in the tofu was physically degraded by heating or the like. When PCR is carried out using such a DNA as a template, amplification efficiency may vary among samples even if the same primers are used, thus making precise quantification difficult.

Modified EPSPS protein gene was used as a target gene. The pair of primers, CP4 FP and CP4 RP (SEQ ID NOS: 7 and 8), and the fluorescence-labeled probe (SEQ ID NO: 9) as described in Example 2 were used for the amplification and detection of this gene.

Soybean actin gene was used as a control gene. The pair of primers, actin FP and actin RP (SEQ ID NOS: 10 and 11), and a fluorescence-labeled probe (SEQ ID NO: 12) as described in Example 2 were used for the amplification and detection of actin gene. FAM and TAMRA were added at the 5′-termini and the 3′-termini of the two fluorescence- labeled probes, respectively.

A PCR was carried out as described in Example 1. The relative initial amounts of DNAs as templates for the target gene and the control gene were determined using a test sample exclusively containing Roundup Ready. The results are shown in Table 7.

TABLE 7
Tofu test sample (containing 4% of Roundup Ready)

| | Amount of | Amount of | |
|---|---|---|---|
| | target gene | control gene | (a)/(b) × 100 |
| Sample no. | (a) | (b) | (%) |
| 1 | 4.25 | 91.2 | 4.66 |
| 2 | 4.29 | 84.6 | 5.07 |
| 3 | 4.18 | 90.9 | 4.60 |
| 4 | 4.22 | 104 | 4.06 |

| | | |
|---|---|---|
| | Mean (%) | 4.60 |
| | Standard deviation (%) | 0.4148 |
| | Coefficient of variation (%) | 9.0 |
| Interval of | Greatest lower bound (%) | 3.6 |
| 99% confidence | Least upper bound (%) | 5.6 |

As shown in Table 7, the content of GMO (Roundup Ready soybean) in the main raw material soybean could be determined for a processed food such as tofu in which the DNA had been degraded. These results suggest that the contents of GMOs in processed food test samples such as fried bean curd, dried bean curd, soybean milk and defatted soybean in which the DNAs are degraded or damaged as observed for tofu can be determined using the method of the present invention.

As described above, the present invention provides a method for quantitatively and precisely determining a content of a heterologous individual in a population of an organism.

Sequence Listing Free Text

SEQ ID NO:1: Designed oligonucleotide primer designated as pat FP to amplify a portion of pat gene.

SEQ ID NO:2: Designed oligonucleotide primer designated as pat RP to amplify a portion of pat gene.

SEQ ID NO:3: Designed oligonucleotide probe to detect pat gene sequence.

SEQ ID NO:4: Designed oligonucleotide primer designated as Zein FP to amplify a portion of zein gene.

SEQ ID NO:5: Designed oligonucleotide primer designated as Zein RP to amplify a portion of zein gene.

SEQ ID NO:6: Designed oligonucleotide probe to detect zein gene sequence.

SEQ ID NO:7: Designed oligonucleotide primer designated as CP4 FP to amplify a portion of EPSPS protein gene.

SEQ ID NO:8: Designed oligonucleotide primer designated as CP4 RP to amplify a portion of EPSPS protein gene.

SEQ ID NO:9: Designed oligonucleotide probe to detect EPSPS protein gene sequence.

SEQ ID NO:10: Designed oligonucleotide primer designated as actin FP to amplify a portion of actin gene.

SEQ ID NO:l1: Designed oligonucleotide primer designated as actin RP to amplify a portion of actin gene.

SEQ ID NO:12: Designed oligonucleotide probe to detect actin gene sequence.

What is claimed is:

1. A method for quantitatively determining a content of a heterologous individual in a population of an organism, characterized in that the method comprises:
a) obtaining two or more samples from a population of an organism or a processed product from the population of the organism;
b) extracting DNAs from the samples;
c) preparing reaction mixtures each containing one of the extracted DNAs, primers specific for a target gene and primers specific for a control gene;
d) subjecting the reaction mixtures to quantitative PCRs to determine amounts of amplification products specific for the target gene and amounts of amplification products specific for the control gene;
e) determining a content of a heterologous individual in the population of the organism based on the amounts of the amplification products specific for the target gene and the amounts of the amplification products specific for the control gene; and
f) determining the confidence of the determined content by statistical analysis.

2. The method according to claim 1, wherein the population of the organism is an unprocessed crop.

3. The method according to claim 2, wherein the unprocessed crop is maize, soybean, rice, wheat, barley, tomato, pumpkin, sweet potato, cotton, rapeseed or beet.

4. The method according to claim 1, wherein the processed product from the population of the organism is a processed food made from a crop as a raw material.

5. The method according to claim 4, wherein the processed food is tofu, tofu-related food, defatted soybean, soybean flour, unpurified soybean protein, corn grits, corn flour, cornstarch, popcorn or snack food.

6. The method according to claim 1, wherein the specific gene is a foreign gene.

7. The method according to claim 6, wherein the foreign gene is pat gene, modified EPSPS gene, CryIA gene, CryIIIA gene or bar gene.

8. The method according to claim 1, wherein the control gene is a gene commonly present in the population of the organism.

9. The method according to claim 8, wherein the control gene is zein gene, actin gene or lectin gene.

10. The method according to claim 1, wherein four to eight samples are obtained in step a) and an interval of 99% confidence is determined by statistical analysis using interval estimation in step f).


SEQUENCE LISTING (12 sequences)

SEQ ID NO: 1 - DNA, 22 bp, Artificial Sequence
  Synthetic
gcgcaaggttttaagtctgtgg

SEQ ID NO: 2 - DNA, 22 bp, Artificial Sequence
  Synthetic
caaagcctcatgcaacctaaca

SEQ ID NO: 3 - DNA, 28 bp, Artificial Sequence
  Synthetic
tgctgttataggccttccaaacgatcca

SEQ ID NO: 4 - DNA, 20 bp, Artificial Sequence
  Synthetic
ttaccgcttcagacgatgcc

SEQ ID NO: 5 - DNA, 20 bp, Artificial Sequence
  Synthetic
cataatctgcgagacggcgt

SEQ ID NO: 6 - DNA, 27 bp, Artificial Sequence
  Synthetic
tgccacagatgatgacgcctaacatga

SEQ ID NO: 7 - DNA, 20 bp, Artificial Sequence
  Synthetic
cgatttcgacagcaccttca

SEQ ID NO: 8 - DNA, 19 bp, Artificial Sequence
  Synthetic
ttccgatttcacctgcacg

SEQ ID NO: 9 - DNA, 22 bp, Artificial Sequence
  Synthetic
tgttgaacccgctgcgcgaaat

SEQ ID NO: 10 - DNA, 19 bp, Artificial Sequence
  Synthetic
ccttcaatgtgcctgccat

SEQ ID NO: 11 - DNA, 20 bp, Artificial Sequence
  Synthetic
cagttgtgcgaccacttgca

SEQ ID NO: 12 - DNA, 28 bp, Artificial Sequence
  Synthetic
tatgtggccatccaagctgttctctcct